(19) United States
(12) Patent Application Publication
(10) Pub. No.: US 2021/0304845 A1
(43) Pub. Date: Sep. 30, 2021

(54) METHOD AND SYSTEM FOR PROCESSING GENETIC DATA

(71) Applicant: Alibaba Group Holding Limited, George Town (KY)

(72) Inventor: Fei Gu, Hangzhou (CN)

(21) Appl. No.: 17/196,728

(22) Filed: Mar. 9, 2021

(30) Foreign Application Priority Data
Mar. 31, 2020 (CN) 202010244886.X

(51) Int. Cl. G16B 30/10; G16H 10/40; G16B 30/00; G16B 30/20; G16B 20/20

(52) U.S. Cl. CPC G16B 30/10; G16H 10/40; G16B 20/20; G16B 30/20; G16B 30/00

(57) ABSTRACT

Processing genetic data is disclosed including inputting data, the data corresponding to source data for gene sequencing on a sample, receiving a selected data processing technique, performing, using the selected data processing technique, gene sequencing processing on the inputted data based on the pathogen genome of a predetermined pathogen to obtain a data processing result, the data processing result indicating whether the sample includes the predetermined pathogen, and presenting the data processing result.

### CROSS REFERENCE TO OTHER APPLICATIONS

This application claims priority to People's Republic of China Patent Application No. 202010244886.X entitled DATA PROCESSING METHOD, MEANS, STORAGE, MEDIUM AND COMPUTING DEVICE filed Mar. 31, 2020 which is incorporated herein by reference for all purposes.

### FIELD OF THE INVENTION

The present invention relates to a method and a system for processing genetic data.

### BACKGROUND OF THE INVENTION

In the event that some new diseases occur, especially some highly infectious diseases, one factor for rapid diagnosis of the diseases is an understanding of the diseases' pathology (e.g., the genetic characteristics of the pathogen (such as a virus) that cause the disease). Therefore, performing gene sequencing of diagnostic samples can be useful and thereby determines whether the diagnostic samples are pathogen infections.

As an example, the novel coronavirus has had a severe impact on the lives and safety of people and on the nation's economic development. Viruses other than the novel coronavirus (for example, the severe acute respiratory syndrome (SARS) associated virus and influenza viruses) also cause enormous harm to the nation and people. Therefore, in the event that serious infectious diseases are to be prevented and controlled, conducting rapid testing and identification of patient samples is needed to determine whether the patient samples include specific viruses and then to perform effective quarantine and treatment on patients having the serious infectious diseases.

Conventionally, the testing used to determine whether a sample is infected with a predetermined pathogen is directed at the genomes of a great number of microbes or pathogens. As a result, the tests suffer from at least long testing times and poor precision.

### BRIEF DESCRIPTION OF THE DRAWINGS

Various embodiments of the invention are disclosed in the following detailed description and the accompanying drawings.

FIG. 1 is a functional diagram illustrating a programmed computer system for processing genetic data in accordance with some embodiments.

FIG. 2 is a flowchart of an embodiment of a process for processing genetic data.

FIG. 3 is a flowchart of another embodiment of a process for processing genetic data.

FIG. 4 is a flowchart of yet another embodiment of a process for processing genetic data.

FIG. 5 is a flowchart of an example of a process for processing genetic data.

FIG. 6 is a scenario application illustration of another example of a process for processing genetic data.

FIG. 7 is a structural block diagram of an embodiment of a system for processing genetic data.

FIG. 8 is a structural block diagram of another embodiment of a system for processing genetic data.

FIG. 9 is a structural block diagram of yet another embodiment of a system for processing genetic data.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Some of the terms that appear in the description of embodiments of the present application are explained below:

Whole genome: All pathogenic microorganisms such as viruses and bacteria have their own genomic compositions, which are typically ribonucleic acid (RNA) or deoxyribonucleic acid (DNA). The whole genome is the sum total of the RNA or DNA.

Gene sequencing: The use of bioreagents and a sequencer to detect genomic fragments and convert the genomic fragments into characters.

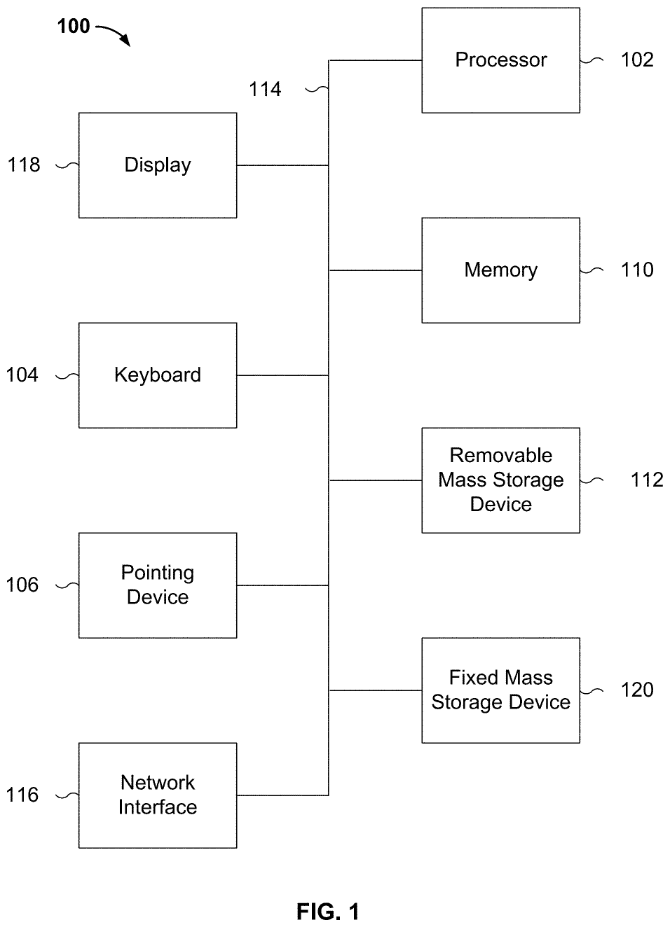

FIG. 1 is a functional diagram illustrating a programmed computer system for processing genetic data in accordance with some embodiments. As will be apparent, other computer system architectures and configurations can be used to process genetic data. Computer system 100, which includes various subsystems as described below, includes at least one microprocessor subsystem (also referred to as a processor or a central processing unit (CPU)) 102. For example, processor 102 can be implemented by a single-chip processor or by multiple processors. In some embodiments, processor 102 is a general purpose digital processor that controls the operation of the computer system 100. Using instructions retrieved from memory 110, the processor 102 controls the reception and manipulation of input data, and the output and display of data on output devices (e.g., display 118).

Processor 102 is coupled bi-directionally with memory 110, which can include a first primary storage, typically a random access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 102. Also as is well known in the art, primary storage typically includes basic operating instructions, program code, data, and objects used by the processor 102 to perform its functions (e.g., programmed instructions). For example, memory 110 can include any suitable computer-readable storage media, described below, depending on whether, for example, data access needs to be bi-directional or uni-directional. For example, processor 102 can also directly and very rapidly retrieve and store frequently needed data in a cache memory (not shown).

A removable mass storage device 112 provides additional data storage capacity for the computer system 100, and is coupled either bi-directionally (read/write) or uni-directionally (read only) to processor 102. For example, storage 112 can also include computer-readable media such as magnetic tape, flash memory, PC-CARDS, portable mass storage devices, holographic storage devices, and other storage devices. A fixed mass storage 120 can also, for example, provide additional data storage capacity. The most common example of mass storage 120 is a hard disk drive. Mass storages 112 and 120 generally store additional programming instructions, data, and the like that typically are not in active use by the processor 102. It will be appreciated that the information retained within mass storages 112 and 120 can be incorporated, if needed, in standard fashion as part of memory 110 (e.g., RAM) as virtual memory.

In addition to providing processor 102 access to storage subsystems, bus 114 can also be used to provide access to other subsystems and devices. As shown, these can include a display monitor 118, a network interface 116, a keyboard 104, and a pointing device 106, as well as an auxiliary input/output device interface, a sound card, speakers, and other subsystems as needed. For example, the pointing device 106 can be a mouse, stylus, track ball, or tablet, and is useful for interacting with a graphical user interface.

The network interface 116 allows processor 102 to be coupled to another computer, computer network, or telecommunications network using a network connection as shown. For example, through the network interface 116, the processor 102 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 102 can be used to connect the computer system 100 to an external network and transfer data according to standard protocols. For example, various process embodiments disclosed herein can be executed on processor 102, or can be performed across a network such as the Internet, intranet networks, or local area networks, in conjunction with a remote processor that shares a portion of the processing. Additional mass storage devices (not shown) can also be connected to processor 102 through network interface 116.

An auxiliary I/O device interface (not shown) can be used in conjunction with computer system 100. The auxiliary I/O device interface can include general and customized interfaces that allow the processor 102 to send and, more typically, receive data from other devices such as microphones, touch-sensitive displays, transducer card readers, tape readers, voice or handwriting recognizers, biometrics readers, cameras, portable mass storage devices, and other computers.

The computer system shown in FIG. 1 is but an example of a computer system suitable for use with the various embodiments disclosed herein. Other computer systems suitable for such use can include additional or fewer subsystems. In addition, bus 114 is illustrative of any interconnection scheme serving to link the subsystems. Other computer architectures having different configurations of subsystems can also be utilized.

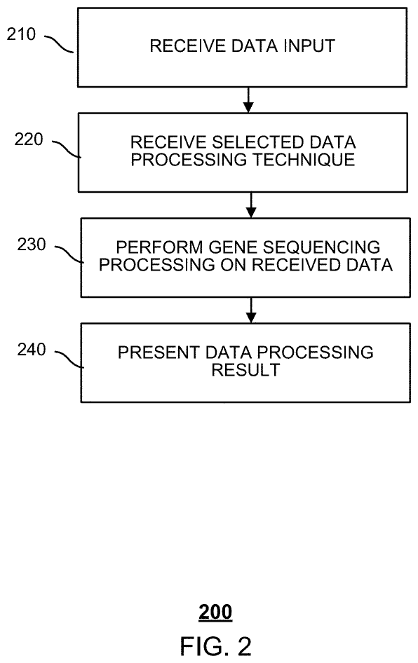

FIG. 2 is a flowchart of an embodiment of a process for processing genetic data. In some embodiments, the process 200 is implemented by the computer system or device 100 of FIG. 1 and comprises:

In 210, the device receives data input. In some embodiments, the data corresponds to source data for gene sequencing on a sample.

In some embodiments, the received data corresponds to data that can be used for gene sequencing. For example, the data is obtained after a sample undergoes sample processing. For example, in determining whether a certain person has contracted a certain pathogen, the sample is directly acquired from a part of the person's body. The sample then undergoes testing by an instrument, which outputs data that is used to characterize sample features. This outputted data can be image data, or the outputted data can be specific data values. As an example, the sample is tested by the instrument, where the instrument outputs image data, the image data includes images of wave crests of various amplitudes, and different amplitudes correspond to different nucleic acids. As a result, the nucleic acid fragments or gene sequences are obtained from the order of the nucleic acids of the inputted data.

In some embodiments, the executing entity that inputs the data here can be a person, or the executing entity can be a predetermined program. In the event that the executing entity is a person, the executing entity can be the person from which the sample was taken, or the executing entity can be a medical professional responsible for the sample.

In some embodiments, prior to the receiving of the data, descriptions of the data processing technique can include one or more of the following: data formatting requirements, data processing price, and/or data processing priority level. Some of the descriptions can be provided to make the operating process more clear. For example, in the event that the data processing technique is being performed, the descriptions of the data processing technique involve formatting requirements or file version issues. Therefore, after the data processing technique has been completed, the descriptions can be provided first to effectively avoid excessively long data processing times. In another example, the data processing technique is paid processing, the data processing price can be indicated before the data processing technique is performed. The data processing price can be based on the amount of data or on the number of times that the data has been processed. The examples of basing pricing on the amount of data or on the amount of processing time are merely examples, and the data processing price can be determined in other ways. In addition, data processing priority levels can be described before the data processing technique is performed. For example, some data is more time-sensitive and requires high priority processing while other data has normal time sensitivity and does not require high priority processing. Therefore, data can be processed differently based on whether the data is labeled with a priority level and based on the priority level of the data.

In 220, the device receives a selected data processing technique.

In some embodiments, prior to the receiving of the selected data processing technique, multiple data processing techniques and the corresponding principles, advantages, and disadvantages are presented. In some embodiments, multiple data processing techniques are presented prior to the performing of the data processing technique. In some embodiments, in the event that multiple data processing techniques are presented, separately the principles, advantages, and disadvantages of the various data processing techniques are also presented. For example, a data processing technique corresponds to distributed processing, or a data processing technique corresponds to parallel processing. In some embodiments, the data processing technique corresponds to the adoption of a specific data processing algorithm or the adoption of an artificial intelligence model. Advantages and disadvantages of a data processing technique can include: advantages—high processing speed, short duration, accurate results, etc., and disadvantages—requires a manual differentiation operation, necessary high expense, lack of smart interaction, etc. Please note that the above advantages and disadvantages are merely examples and other advantages or disadvantages are not excluded.

In 230, the device performs, based on the selected data processing technique, gene sequencing processing on the received data based on a pathogen genome of a predetermined pathogen to obtain a data processing result indicating whether the sample includes the predetermined pathogen.

The targeted predetermined pathogen can be a single pathogen. For example, the targeted predetermined pathogen can comprise a novel coronavirus or a severe acute respiratory syndrome (SARS) associated coronavirus. The use of a single pathogen for data processing has the advantages of speed and accuracy relative to conventional data processing techniques, in which multiple pathogens need to be individually aligned. Conventionally, because there are many pathogens in the gene sequencing system, in order to determine, which pathogen, the sample belongs to, the sample is compared with each pathogen of the multiple pathogens, which is slower than the gene sequencing system of the present application because the present application can use a single pathogen.

In some embodiments, the performing of the gene sequencing processing on the received data based on the pathogen genome of a predetermined pathogen to obtain a data processing result indicating whether the sample includes the predetermined pathogen entails multiple approaches. As an example, one approach is as follows: converting the data to a gene sequence format to obtain reads, the reads corresponding with gene sequence fragments; comparing the reads to the pathogen genome of the predetermined pathogen; determining whether the reads are included in the pathogen genome; assembling the reads into a sample genome; and by aligning the sample genome with the pathogen genome, obtaining a data processing result indicating whether the sample includes the predetermined pathogen.

In 240, the device presents the data processing result.

In some embodiments, in the event that the data processing result is presented, different ways of presenting the data processing result exist due to the fact that the data processing result can vary.

For example, in the event that the data processing result is presented, the data processing result can be expressed as a degree of similarity between the sample genome and the pathogen genes, or the data processing result can be expressed as a probability that the sample includes the predetermined pathogen. Therefore, presenting the data processing result can include: presenting the probability that the sample includes the predetermined pathogen, the probability being expressed in the form of a numerical value or graphically. In addition, the data processing result can further comprise: whether variations have occurred in the predetermined pathogen. Therefore, the presenting of the data processing result can further comprise: variations that have occurred in the predetermined pathogen and the positions of the variations. As an example, in the event that variations occur in a predetermined pathogen, the places that differ from the genome of the predetermined pathogen can be labeled. In the event that the quantity of labeled positions satisfies a predetermined number, it can be determined that variations have occurred in the predetermined pathogen. When labeling, different colors can be used to display the places, or special marks can be used to circle the places.

In some embodiments, the process 200 further comprises: generating and printing a report, the report including the data processing result. The report can take multiple forms. For example, the report can be a traditional paper report or an electronic one.

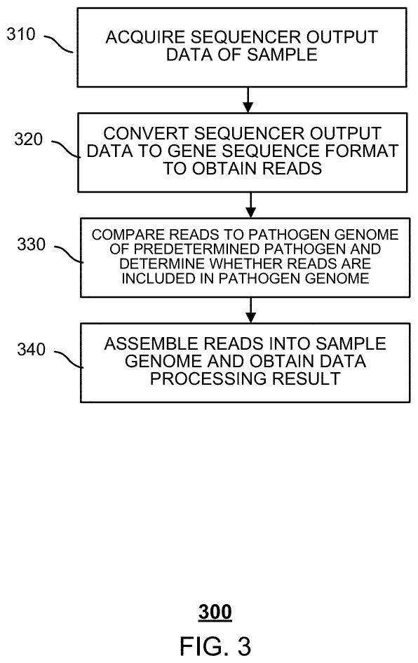

FIG. 3 is a flowchart of another embodiment of a process for processing genetic data. In some embodiments, the process 300 is implemented by the computer system or device 100 of FIG. 1 and comprises:

In 310, the device acquires sequencer output data of the sample, the sequencer output data being data resulting from sequencing performed on the sample by a sequencer.

In some embodiments, the sequencer output data is data resulting from sequencing performed on the sample by a sequencer. Sequencers can also be called gene sequencers. The sequencers can be instruments that determine the base sequences, categories, and quantities of RNA or DNA fragments. In the event that a sample is sequenced by a sequencer, the sequencing result that is output can be a series of images. In other words, the sequencer output data can correspond to a series of images.

In some embodiments, in the event that sequencer output data is acquired for a sample, multiple approaches may be taken. For example, the sequencer output data is acquired in different ways depending on different sequencing techniques or different sequencing parameters. As an example, acquiring sequencer output data for a sample includes at least one of the following:

Acquiring sequencer output data for a sample through single-end sequencing: Single-end sequencing is gene sequencing that begins from only one end of a chromosome. Single-end sequencing is one of the simplest sequencing techniques. This sequencing technique can quickly and economically provide large volumes of high-quality sequencer output data.

Acquiring sequencer output data for a sample through paired-end sequencing: Paired-end sequencing is sequencing that is performed from both ends of a chromosome and that generates high-quality, alignable sequence data. As an aspect, paired-end sequencing can be helpful in detecting genome shuffling, repeated sequence elements, fusion genes, and new transcripts.

Acquiring sequencer output data for a sample by using different sequence lengths to conduct sequencing: Sequence lengths refer to a length of one sequencing run in the event that sequencing a chromosome is performed. The length can be a suitable sequence length selected based on sample type, application, coverage requirements, or any combination thereof

It should be noted that the different acquisition techniques described above can be selected flexibly based on differences in specific sample types and sample applications.

In 320, the device converts the sequencer output data to a gene sequence format to obtain reads. In some embodiments, the output data of the sequencer corresponds to an image of wave crests of different amplitudes, the different amplitudes represent different nucleic acids, and the reads are obtained based on the order of the represented nucleic acids.

In some embodiments, in the event that a sample is sequenced by a sequencer, the sequencing result that is output is a series of images. In the event that a sample undergoes gene sequencing, sequence alignments between sample genes and genes of the pathogen are quickly and conveniently performed by a sequence formatting technique. Therefore, prior to performing gene sequencing alignments, the sample sequencer output data to be aligned can be converted to a gene sequence format to obtain reads. By adopting the technique of converting sequencer output data to a gene sequence format to obtain reads effectively, the convenience and efficiency of subsequent gene alignments can be increased.

In some embodiments, in the event that the sequencer output data corresponds to a series of images, this series of images is to correspond to gene fragments of the sample. Therefore, in the event that the sequencer output data for the sample undergoes gene sequence format conversion, the result is reads corresponding to the series of images included in the sample. In other words, multiple images individually correspond to multiple reads. These multiple reads together compose the entire genome of the sample.

In 330, the device compares the reads to the pathogen genome of the predetermined pathogen and determines whether the reads are included in the pathogen genome.

In some embodiments, the predetermined pathogen corresponds to a single pathogen. For example, the predetermined pathogen includes a novel coronavirus or a severe acute respiratory syndrome (SARS) associated coronavirus. In the event that the predetermined pathogen is a single pathogen, then determining whether reads are included in the pathogen genome is all that remains to be performed in the event that the reads are compared to the pathogen genome. There is no need to align the reads with multiple pathogen genomes in a one by one polling approach to determine whether the reads exist in the genomes of multiple pathogens. Thus, the alignment and determination workloads are significantly reduced.

In some embodiments, multiple approaches exist that are adopted when implementing the following: comparing reads to a pathogen genome and determining whether the reads are included in the pathogen genome. For example, a distributed processing technique and/or a parallel processing technique can be employed to compare the reads to the pathogen genome and determine whether the reads are included in the pathogen genome. In some embodiments, the distributed processing technique comprises: distributing a plurality of reads of the sample across multiple pieces of server hardware for processing. In some embodiments, the parallel processing technique comprises: putting a plurality of reads of the sample in multiple tasks of a single piece of server hardware for parallel processing. With the distributed processing technique and/or the parallel processing technique described above, not only is it possible to make more efficient use of computing resources, but it is also possible to further accelerate the sequencing process and thus increase sequencing speed. It should be noted that the distributed processing and parallel processing techniques can be selected singly or in combination. The distributed processing and parallel processing techniques can be used flexibly based on the quantity of samples to be sequenced and specific applications of the samples. In addition, the pathogen genome corresponds to the predetermined genome. In other words, the pathogen genome corresponds to the genome of the predetermined pathogen. Therefore, it can be determined through a comparison of the sample genome with the pathogen genome whether the sample has been infected with the predetermined pathogen.

In some embodiments, after comparing reads to a pathogen genome and determining whether the reads are included in the pathogen genome, and upon determining that reads are included in the pathogen genome, the positions of the reads in the pathogen genome are determined. In other words, specific positions of the reads are determined in the pathogen genome. After the reads are located and the determined positions are labeled, displaying the positions of the reads and analyzing these reads can be easily performed.

In 340, the device assembles the reads into a sample genome, and by aligning or comparing the sample genome with the pathogen genome, obtains a data processing result indicating whether the sample includes the predetermined pathogen. Regarding the alignment or comparing, a threshold can be preset, for example, the threshold can be set to 90% alignment. In event that the sample is aligned at or above the threshold of 90%, the sample is determined to include the predetermined pathogen, because only 10% of the sample genome is different from the target pathogen genome.

In some embodiments, the distributed processing technique and/or parallel processing technique described above is adopted when assembling reads into a sample genome. Please note that each operation in the entire gene sequencing process can utilize the distributed processing technique and/or the parallel processing technique, which were described above. The adoption of the distributed processing technique and/or the parallel processing technique by operations 330 and 340 in the above description were merely an example.

In some embodiments, after the reads are assembled into a sample genome, the assembled sample genome can be aligned with the pathogen genome to determine a degree of similarity between the sample genome and the pathogen genome.

This degree of similarity can be expressed in various forms. For example, the degree of similarity is expressed as a percentage, e.g., the degree of similarity between the sample genome and the pathogen genome is 80%. In other words, 80% of the reads in the sample genome are included in the pathogen genome. In another example, the degree of similarity is expressed by using numerical values indicating similarity and dissimilarity. For example, 1 indicates that the sample genome and the pathogen genome are similar, while 0 indicates that the sample genome and the pathogen genome are dissimilar. A threshold value can be used to differentiate between similar and dissimilar genomes. In the event that the number of reads of the sample found in the pathogen genome exceeds the threshold value, the sample genome is determined to be similar to the pathogen genome. Otherwise, the sample genome is determined to be dissimilar to the pathogen genome.

By determining the degree of similarity between the sample genome and the pathogen genome, determining directly whether the sample has been infected by the pathogen based on the degree of similarity is possible. Of course, determining whether the sample has been infected by the pathogen based on the degree of similarity requires that a degree of similarity threshold can be provided. In the event that the degree of similarity between the sample genome and the pathogen genome satisfies the degree of similarity threshold, a determination can be made that the sample is infected with the pathogen. In the event that the degree of similarity between the sample genome and the pathogen genome does not satisfy the degree of similarity threshold, the determination can be made that the sample is not infected with the pathogen.

In some embodiments, variant sites of the predetermined pathogen are determined by comparing the sample genome to the pathogen genome. Within the reads, the positions where the sequences differ between the sample genome and the pathogen genome can be variant sites of the predetermined pathogen. Please note that these variant sites can be the positions in the reads where the sample genome and the pathogen genome are found to differ after a determination that the degree of similarity between the sample genome and the pathogen genome is greater than a fixed value. Since gene variations are uncommon, in some embodiments, this value can be relatively high to effectively avoid incorrect determination of variant sites.

The above embodiments take the approach of aligning the reads of a sample with the genome of a predetermined pathogen. Via comparison with the genome of only a single predetermined pathogen, a determination whether the sample is infected with the predetermined pathogen can be made. Thus, a fast and precise determination of whether a sample is infected with a predetermined pathogen can be made, and the long testing times and the poor testing precision resulting from the fact that the testing used to determine whether a sample is infected with a predetermined pathogen is always directed at the genomes of a large number of pathogens.

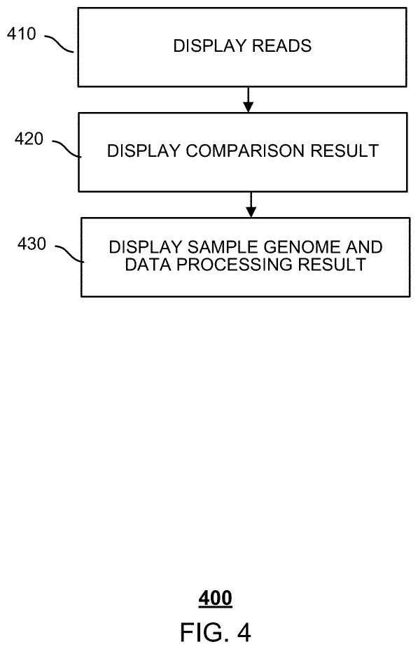

FIG. 4 is a flowchart of yet another embodiment of a process for processing genetic data. In some embodiments, the process 400 is implemented by the computer system or device 100 of FIG. 1 and comprises:

In 410, the device displays reads, the reads being obtained by converting sequencer output data of a sample to a gene sequence format, and the sequencer output data corresponding to data resulting from sequencing performed on the sample by a sequencer.

In 420, the device displays a comparison result, the comparison result indicating whether the reads are included in the pathogen genome of the predetermined pathogen.

In 430, the device displays the sample genome and the data processing result indicating whether the sample includes the predetermined pathogen, the sample genome being assembled from the reads.

By displaying each operation in the data processing operation 400, not only can a fast and precise determination of whether a sample is infected with a predetermined pathogen be obtained, but the long testing times and poor testing precision resulting from the testing used to determine whether a sample is infected with a predetermined pathogen always is directed at the genomes of a great number of pathogens or microbes. Moreover, the data processing process 400 achieves user-friendly interaction leading to more convenient operation.

In some embodiments, in the process of gene sequencing implemented through user-friendly interactions, instructions can be input based on simple user operations of a keyboard and/or a mouse and in response to these instructions, corresponding results can be output.

As an example: receiving a first instruction, the first instruction being for requesting display of read positions in the pathogen genome; in response to the receiving of the first instruction, displaying the read positions in the pathogen genome in the event that the reads are included in the pathogen genome. The receiving of the first instruction can be implemented by receiving an operation of a first button on an application interface via a keyboard and/or a mouse. In other words, the user can, through the first button on the application interface, obtain the read positions (which were determined in the background) in the pathogen genome.

In another example: receiving a second instruction, the second instruction being for requesting display of a degree of similarity between the sample genome and the pathogen genome; in response to the receiving of the second instruction, displaying the degree of similarity between the sample genome and the pathogen genome and a probability that the sample includes the predetermined pathogen. Correspondingly, in the event that an operation of a second button on an application interface via a keyboard and/or a mouse is received, the background acquires the request content corresponding to the second button and then displays the degree of similarity between the sample genome and the pathogen genome and the probability that the sample includes the predetermined pathogen on the application interface.

In yet another example: receiving a third instruction, the third instruction being for requesting display of variant sites of the predetermined pathogen; in response to the receiving of the third instruction, displaying variant sites of the predetermined pathogen by comparing the sample genome to the pathogen genome. Correspondingly, in the event that an operation of a third button on an application interface via a keyboard and/or a mouse is received, the background acquires the request content corresponding to the third button and then displays the variant sites of the predetermined pathogen on the application interface.

In some embodiments, in the gene sequencing process using user-friendly interactions, feedback is generated directly in a consolidated report, without the user having to input instructions in the gene sequencing process, for some information that is more important or critical. For example: receiving a request list, the request list comprising sequencing result items for which feedback is requested; generating and displaying a report in response to the request list, the report comprising sequencing results for items included in the request list. Receiving a request list can be a default setting in the event that the sequencer output data for the sample is input into the device. In other words, all that is needed is inputting the sequencer output data to generate a report. Moreover, the report is to include the sequencing results for the request items in the request list. Of course, to make report generation more flexible, the items in the request list can be set as optional. In this way, the user can flexibly select needed items as necessary and later obtain the sequencing results for the needed items.

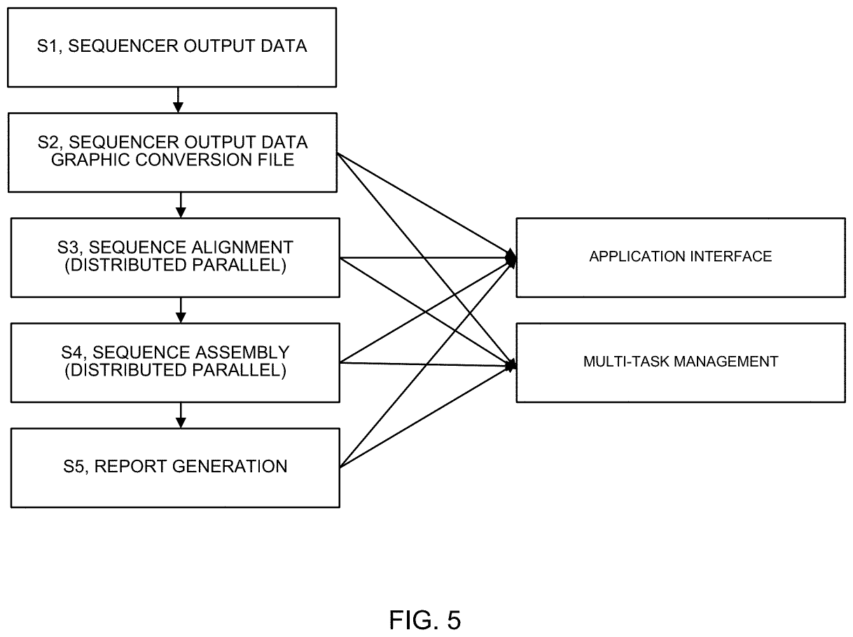

FIG. 5 is a flowchart of an example of a process for processing genetic data. In some embodiments, the process 500 comprises:

S1: acquires sequencer output data of the sample, the sequencer output data being data from sequencing performed on the sample by a sequencer.

S2, Sequencer Output Data Graphic Conversion File: The sequencer output data from the sequencer corresponds to a series of images. These images are to be converted into a gene sequencing format. In other words, the series of images are converted into a file comprising reads corresponding to the images. These reads are in the gene sequencing format.

S3, Sequence Alignment: The sequencing data corresponds to a series of reads, which are to be compared to the pathogen genome to determine whether the sequencing data matches the sequence of the pathogen genome and at which positions of the pathogen genome.

S4, Sequence Assembly: The sequencing data reads are assembled into a long genome (i.e., the sample genome) to facilitate subsequent analysis, e.g., degree of similarity with the pathogen genome, presence/absence of variations, etc.

S5, Report Generation: Based on instructions input by the user, a report is generated comprising sequencing results corresponding to items requested by the user or a report is generated comprising sequencing results corresponding to items set by default.

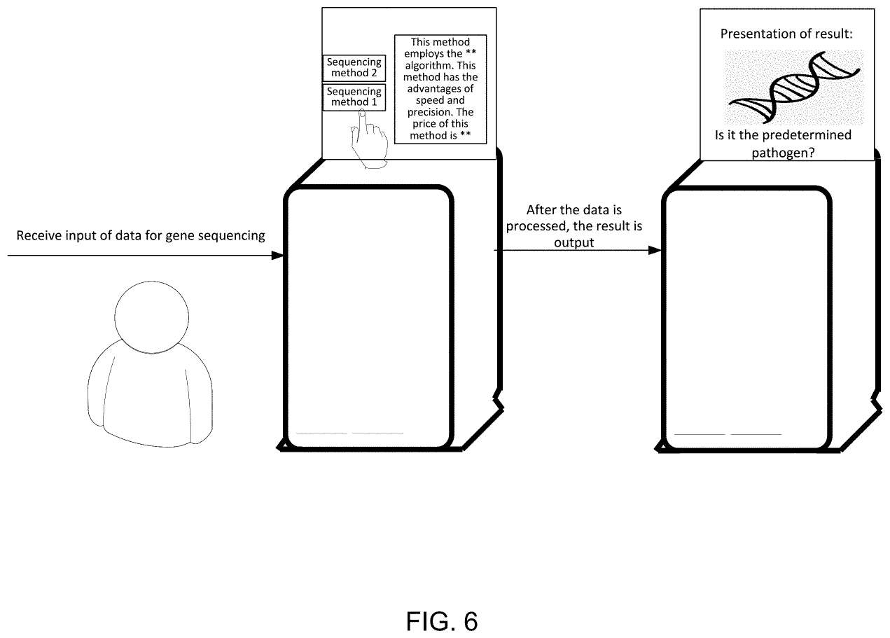

FIG. 6 is a scenario application illustration of another example of a process for processing genetic data. In some embodiments, as long as sequencer output data has been input, the user can obtain feedback results as requested upon inputting operating instructions through an application interface. For example, the user, using a data transmission device, inputs data from sequencing performed on a sample by a sequencer (for example, the user, if a patient, enters data obtained from the patient's own sample). Then, through prompts on the interface, the user inputs instructions that are of importance to himself or herself and then browses on the interface for the results. For example, sequencing results are displayed on the application interface, and the following result is displayed: is the user infected with a predetermined pathogen (e.g., novel coronavirus (COVID-19))? In the event that the corresponding results are being displayed, the corresponding results can also be marked with different colors. For example, red indicates COVID-19, and green indicates that it is not COVID-19. Since different colors are very noticeable, the use of color markings enables immediate comprehension of the feedback results.

In some embodiments, new data is processed using artificial intelligence based on large volumes of already processed data or already identified samples. For example, the process can be implemented using artificial intelligence: collecting data used to sequence a sample; obtaining, using a data processing model, data processing results corresponding to the collected data, the data processing model being trained using machine learning of multiple sets of data; each set of the multiple sets of data comprises: data for gene sequencing and data processing results corresponding to the data. Please note that data used to sequence a sample can be images directly obtained from a sequencer or can be numerical data obtained after converting the sequencer images. Of course, in the event that the sequencer is made a part of the artificial intelligence data processing, the sample itself can directly serve as data for processing. Multiple types of data processing results corresponding to the data can exist. For example, the multiple types of data processing results are gene sequences directly obtained by sequencing, or the multiple types of data processing results are comparison results obtained after performing a comparison with the predetermined pathogen. The multiple types of data processing results also can be the probabilities of being regarded as infected with the predetermined pathogen.

In addition, the sample data used to train the data processing model can originate from multiple sources. For example, the sample data is collected directly from hospitals, or the sample data is obtained from corresponding testing organizations. The sample data can also be collected from personnel who participated in genetic testing of the pathogen. The more complete the collected sample data is, the more ideal the data processing model is that results from training. Therefore, the data processing results with such a data processing model can be more accurate.

In some embodiments, the present application supports the end-to-end processing flow, from the sequencer output data to generation of the test report. The sequence alignment and sequence assembly portions of the processing flow support parallel analysis or distributed analysis depending on differences in server hardware. The processing process has a good human-machine interface for the convenience of testing personnel. The processing process also has multi-task management functions for managing analysis, termination, re-analysis, and other such operations involving multiple tasks.

The various embodiments of the data processing process of the present invention have at least the following advantages over conventional data processing processes:

(1) This data processing process can test for a predetermined pathogen. Therefore, the data processing process can test both quickly and precisely. For example, for a set of samples, the analysis can be performed and the report can be typically generated within one half hour. In the event that sample preparation and sequencing time are added, then the analysis can be output after just a few hours.

(2) The use of distributed and parallel whole-genome processing techniques for pathogens can result in more efficient utilization of computing resources and an even faster analysis technique.

(3) By including a sequence assembly process, the sequence assembly process can fully assemble a pathogen genome so that variant sites can be identified. In other words, the sequence assembly process can make the discovery of variant sites convenient.

(4) Using user-friendly interactions and multi-task management can make operation simpler.

(5) Support of multiple kinds of sequencers and sequencing patterns (such as single-end sequencing, paired-end sequencing, and different sequence lengths) can make sequencing more flexible and capable of meeting multiple sequencing requirements.

(6) This data processing process can have a complete development design. Moreover, using keyboard and mouse operations, there is no need to issue commands. Testing personnel can find this data processing process easy to use.

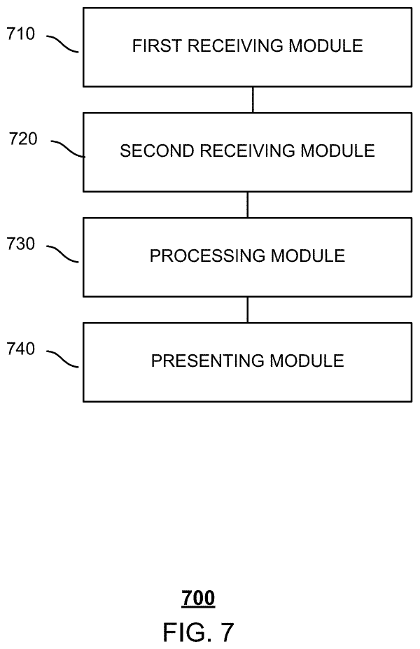

FIG. 7 is a structural block diagram of an embodiment of a system for processing genetic data. In some embodiments, the system 700 is configured to implement the process 200 of FIG. 2 and comprises: a first receiving module 710, a second receiving module 720, a processing module 730, and a presenting module 740.

In some embodiments, the first receiving module 710 is configured to receive data input, the data being source data for gene sequencing on a sample.

In some embodiments, the second receiving module 720, connected to the first receiving module 710, is configured to receive a selected data processing technique.

In some embodiments, the processing module 730, connected to the second receiving module 720, is configured to perform, using the selected data processing technique, gene sequencing processing on the received data based on a pathogen genome of a predetermined pathogen to obtain a data processing result indicating whether the sample includes the predetermined pathogen.

In some embodiments, the presenting module 740, connected to the processing module 730, is configured to present the data processing result.

Please note that the first receiving module 710, the second receiving module 720, the processing module 730, and the presenting module 740 can correspond to operations 210, 220, 230, and 240 of FIG. 2.

The modules described above can be implemented as software components executing on one or more general purpose processors, as hardware such as programmable logic devices and/or Application Specific Integrated Circuits designed to perform certain functions or a combination thereof. In some embodiments, the modules can be embodied by a form of software products which can be stored in a nonvolatile storage medium (such as optical disk, flash storage device, mobile hard disk, etc.), including a number of instructions for making a computer device (such as personal computers, servers, network equipment, etc.) implement the methods described in the embodiments of the present invention. The modules may be implemented on a single device or distributed across multiple devices. The functions of the modules may be merged into one another or further split into multiple sub-modules.

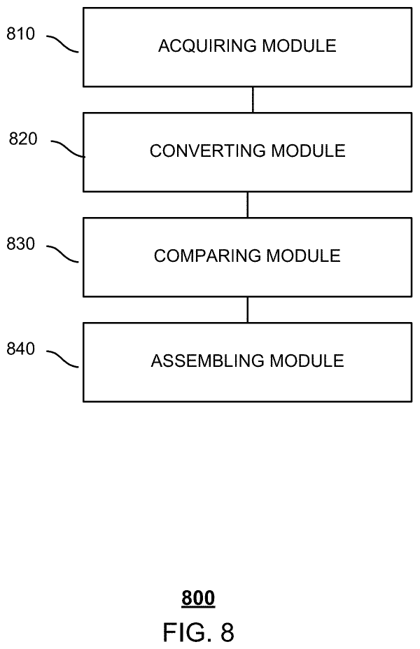

FIG. 8 is a structural block diagram of another embodiment of a system for processing genetic data. In some embodiments, the system 800 is configured to implement the process 300 of FIG. 3 and comprises: an acquiring module 810, a converting module 820, a comparing module 830, and an assembling module 840.

In some embodiments, the acquiring module 810 is configured to acquire sequencer output data of the sample, the sequencer output data being data resulting from sequencing performed on the sample by a sequencer.

In some embodiments, the converting module 820, connected to the acquiring module 810, is configured to convert the sequencer output data to a gene sequence format to obtain reads.

In some embodiments, the comparing module 830, connected to the converting module 820, is configured to compare the reads to a pathogen genome and determine whether the reads are included in the pathogen genome of a predetermined pathogen.

In some embodiments, the assembling module 840, connected to the comparing module 830, is configured to assemble the reads into a sample genome and, by aligning the sample genome with the pathogen genome, obtain a data processing result indicating whether the sample includes the predetermined pathogen.

Please note that the acquiring module 810, the converting module 820, the comparing module 830, and the assembling module 840 can correspond to operations 310, 320, 330, and 340 of FIG. 3.

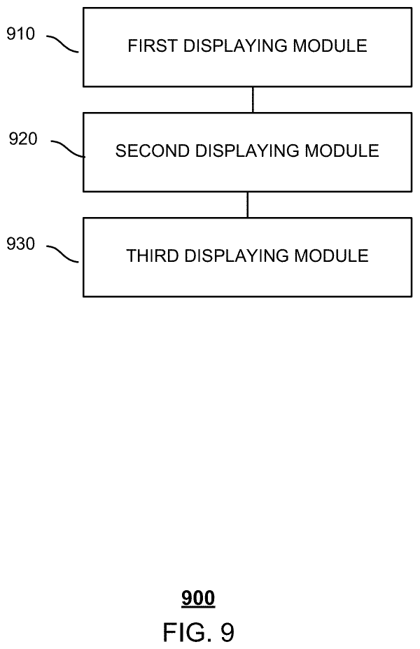

FIG. 9 is a structural block diagram of yet another embodiment of a system for processing genetic data. In some embodiments, the system 900 is configured to implement the process 400 of FIG. 4 and comprises: a first displaying module 910, a second displaying module 920, and a third displaying module 930.

In some embodiments, the first displaying module 910 is configured to display reads, the reads being obtained by converting sequencer output data of the sample to a gene sequence format, and the sequencer output data being data resulting from sequencing performed on the sample by a sequencer.

In some embodiments, the second displaying module 920, connected to the first displaying module 910, is configured to display a comparison result, the comparison result being used to indicate whether the reads are included in the pathogen genome of the predetermined pathogen.

In some embodiments, the third displaying module 930, connected to the second displaying module 920, is configured to display the sample genome and the data processing result indicating whether the sample includes the predetermined pathogen, the sample genome being assembled from the reads.

Please note that the first displaying module 910, the second displaying module 920, and the third displaying module 930 can correspond to operations 410, 420, and 430 of FIG. 4.

In some embodiments, a method is provided. The method includes displaying reads, wherein the reads are obtained by converting sequencer output data of a sample to a gene sequence format, and wherein the sequencer output data corresponds to data resulting from sequencing performed on the sample by a sequencer, displaying a comparison result, wherein the comparison result indicates whether the reads are included in the pathogen genome of the predetermined pathogen, and displaying the sample genome and data processing result indicating whether the sample includes the predetermined pathogen, wherein the sample genome is assembled from the reads.

In some embodiments, the method further comprises: receiving a first instruction, wherein the first instruction requests display of read positions of the reads in the pathogen genome, and in response to the receiving of the first instruction and upon determining that the reads are included in the pathogen genome, displaying the positions of the reads in the pathogen genome.

In some embodiments, the method further comprises: receiving a second instruction, wherein the second instruction requests display of a degree of similarity between the sample genome and the pathogen genome, and in response to the receiving of the second instruction, displaying the degree of similarity between the sample genome and the pathogen genome and a probability that the sample includes the predetermined pathogen.

In some embodiments, the method further comprises: receiving a third instruction, wherein the third instruction requests display of variant sites of the predetermined pathogen, and in response to the receiving of the third instruction, displaying variant sites of the predetermined pathogen based on a comparison of the sample genome to the pathogen genome.

In some embodiments, the method further comprises: receiving a request list, wherein the request list comprises sequencing result items for which feedback is requested, and generating and displaying a report in response to the receiving of the request list, wherein the report comprises sequencing results for the items included in the request list.

In some embodiments, a system is provided. The system includes a processor, and a memory coupled with the processor, wherein the memory is configured to provide the processor with instructions which when executed cause the processor to: acquire sequencer output data of a sample, wherein the sequencer output data corresponds to data resulting from sequencing performed on the sample by a sequencer, convert the sequencer output data to a gene sequence format and obtaining reads based on the gene sequence format, compare the reads to the pathogen genome of the predetermined pathogen, determine whether the reads are included in the pathogen genome based on the comparison, assemble the reads into a sample genome, and by aligning the sample genome with the pathogen genome, obtain a data processing result indicating whether the sample includes the predetermined pathogen.

In some embodiments, a system is provided. The system includes a processor, and a memory coupled with the processor, wherein the memory is configured to provide the processor with instructions which when executed cause the processor to: display reads, wherein the reads are obtained by converting sequencer output data of a sample to a gene sequence format, and wherein the sequencer output data is data resulting from sequencing performed on the sample by a sequencer, display a comparison result, wherein the comparison result indicates whether the reads are included in the pathogen genome of the predetermined pathogen, and display the sample genome and data processing result indicating whether the sample includes the predetermined pathogen, wherein the sample genome is assembled from the reads.

In some embodiments, a computer program product is provided. The computer program product comprises computer instructions for acquiring sequencer output data of a sample, wherein the sequencer output data corresponds to data resulting from sequencing performed on the sample by a sequencer, converting the sequencer output data to a gene sequence format and obtaining reads based on the gene sequence format, comparing the reads to the pathogen genome of the predetermined pathogen, determining whether the reads are included in the pathogen genome based on the comparison, assembling the reads into a sample genome; and by aligning the sample genome with the pathogen genome, obtaining a data processing result indicating whether the sample includes the predetermined pathogen.

In some embodiments, a computer program product is provided. The computer program product comprises computer instructions for displaying reads, wherein the reads are obtained by converting sequencer output data of a sample to a gene sequence format, and wherein the sequencer output data is data resulting from sequencing performed on the sample by a sequencer, displaying a comparison result, wherein the comparison result indicates whether the reads are included in the pathogen genome of the predetermined pathogen, and displaying the sample genome and data processing result indicating whether the sample includes the predetermined pathogen, wherein the sample genome is assembled from the reads.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method, comprising:
inputting data, wherein the data corresponds to source data for gene sequencing on a sample;
receiving a selected data processing technique;
performing, using the selected data processing technique, gene sequencing processing on the inputted data based on a pathogen genome of a predetermined pathogen to obtain a data processing result, wherein the data processing result indicates whether the sample includes the predetermined pathogen; and
presenting the data processing result.

2. The method as described in claim 1, wherein prior to the inputting of the data, the method further comprises:
presenting descriptions of the selected data processing technique, wherein the descriptions include one or more of the following: data formatting requirements, data processing is price, and/or data processing priority level.

3. The method as described in claim 1, wherein prior to the receiving of the selected data processing technique, the method further comprises:
presenting a plurality of data processing techniques and the corresponding principles, advantages, and disadvantages of the plurality of data processing techniques.

4. The method as described in claim 1, wherein the performing of the gene sequencing processing on the inputted data comprises:
converting the data to a gene sequence format to obtain reads;
comparing the reads to the pathogen genome of the predetermined pathogen;
determining whether the reads are included in the pathogen genome;
assembling the reads into a sample genome; and
by aligning the sample genome with the pathogen genome, obtaining the data processing result, wherein the data processing result indicates whether the sample includes the predetermined pathogen.

5. The method as described in claim 1, wherein the presenting of the data processing result comprises:
presenting a probability that the sample includes the predetermined pathogen.

6. The method as described in claim 5, wherein the presenting of the data processing result comprises:
presenting variant sites of the predetermined pathogen.

7. The method as described in claim 1, further comprising:
generating and printing a report including the data processing result.

8. A method, comprising:
acquiring sequencer output data of a sample, wherein the sequencer output data corresponds to data resulting from sequencing performed on the sample by a sequencer;
converting the sequencer output data to a gene sequence format to obtain reads;
comparing the reads to the pathogen genome of the predetermined pathogen;
determining whether the reads are included in the pathogen genome based on the comparison;
assembling the reads into a sample genome; and
by aligning the sample genome with the pathogen genome, obtaining a data processing result indicating whether the sample includes the predetermined pathogen.

9. The method as described in claim 8, wherein:
the method employs a distributed processing technique and/or a parallel processing technique to compare the reads to the pathogen genome of the predetermined pathogen and determine whether the reads are included in the pathogen genome;
the distributed processing technique comprises distributing a plurality of reads of the sample across a plurality of pieces of server hardware for processing; and
the parallel processing technique comprises: putting a plurality of reads of the sample in a plurality of tasks of a single piece of server hardware for parallel processing.

10. The method as described in claim 9, wherein the method employs the distributed processing technique and/or the parallel processing technique to assemble the reads into the sample genome.

11. The method as described in claim 8, further comprising:
upon determining that the reads are included in the pathogen genome, determining positions of the reads in the pathogen genome.

12. The method as described in claim 8, further comprising:
performing at least one of the following:
determining a degree of similarity between the sample genome and the pathogen genome and obtaining a probability that the sample includes the predetermined pathogen; and/or
determining variant sites of the predetermined pathogen based on a comparison of the sample genome to the pathogen genome.

13. The method as described in claim 8, wherein the acquiring of the sequencer output data of the sample comprises:
performing one or more of the following:
acquiring the sequencer output data of the sample through single-end sequencing;
acquiring the sequencer output data of the sample through paired-end sequencing; and/or
acquiring the sequencer output data of the sample through sequencing that uses different sequence lengths.

14. The method as described in claim 8, wherein the predetermined pathogen comprises a novel coronavirus or a severe acute respiratory syndrome (SARS) associated coronavirus.

15. A system, comprising:
a processor; and
a memory coupled with the processor, wherein the memory is configured to provide the processor with instructions which when executed cause the processor to:
input data, wherein the data corresponds to source data for gene sequencing on a sample;
receive a selected data processing technique;
perform, using the selected data processing technique, gene sequencing processing on the inputted data based on the pathogen genome of a predetermined pathogen to obtain a data processing result, wherein the data processing result indicates whether the sample includes the predetermined pathogen; and
present the data processing result.

16. A computer program product being embodied in a tangible non-transitory computer readable storage medium and comprising computer instructions for:
input data, wherein the data corresponds to source data for gene sequencing on a sample;
receiving a selected data processing technique;
performing, using the selected data processing technique, gene sequencing processing on the inputted data based on the pathogen genome of a predetermined pathogen to obtain a data processing result, wherein the data processing result indicates whether the sample includes the predetermined pathogen; and
presenting the data processing result.
